(19) United States
(12) Patent Application Publication
(10) Pub. No.: US 2016/0237461 A1
(43) Pub. Date: Aug. 18, 2016

(54) METHODS FOR BIOSYNTHESIZING METHACRYLATE

(71) Applicant: INVISTA TECHNOLOGIES S.A.R.L., St. Gallen (CH)

(72) Inventors: Adriana Leonora Botes, East Cleveland (GB); Alex Van Eck Conradie, Cleveland (GB)

(73) Assignee: INVISTA TECHNOLOGIES S.A.R.L., St. Gallen (CH)

(21) Appl. No.: 15/051,989

(22) Filed: Feb. 24, 2016

Related U.S. Application Data
(63) Continuation of application No. PCT/US2014/053222, filed on Aug. 28, 2014.
(60) Provisional application No. 61/871,070, filed on Aug. 28, 2013.

(51) Int. Cl. C12P 7/40; C08F 20/10; C12N 15/52; C12N 9/02; C12N 9/10

(52) U.S. Cl. CPC C12P 7/40; C12N 9/0008; C12N 9/13; C12N 9/001; C12N 15/52; C08F 20/10; C12Y 102/01039; C12Y 208/03001; C12Y 103/99

(57) ABSTRACT

This document describes biochemical pathways for producing methacrylate from precursors such as pyruvate via isobutyraldehyde and isobutyryl-CoA, using enzymes such as one or more thioesterases, transferases, or dehydrogenases, as well as recombinant hosts expressing one or more of such enzymes.

This patent application is a continuation of PCT/US2014/053222 filed Aug. 28, 2014 which claims the benefit of priority from U.S. Provisional Application Ser. No. 61/871,070, filed Aug. 28, 2013, teachings of both of which are herein incorporated by reference in their entirety.

### TECHNICAL FIELD

This invention relates to methods for biosynthesizing methacrylate, and more particularly to synthesizing methacrylate using one or more isolated enzymes such as one or more thioesterases, dehydrogenases, or transferases, or using recombinant host cells expressing one or more of such enzymes.

### BACKGROUND

Methacrylate (2-methylpropenoic acid) is an intermediate that is widely used for producing methacrylate esters such as methyl methacrylate or poly(methyl methacrylate), which are used for producing transparent polymethyl methacrylate acrylic plastics. Methacrylate can be synthesized by various routes, including by conversion of acetone cyanohydrin to methacrylamide sulfate using sulfuric acid and hydrolyzation to methacrylic acid, by the oxidation of isobutylene to methacrolein, which is oxidized to methacrylic acid, or by dehydrogenation of isobutyric acid. See, e.g., Bauer, “Methacrylic Acid and Derivatives” in Ullmann's Encyclopedia of Industrial Chemistry 2002, Wiley-VCH, Weinheim. Accordingly, there is a need for biosynthetic methods for producing methacrylate to avoid the use of hazardous chemicals.

### SUMMARY

This disclosure is based at least in part on the development of enzymatic systems and recombinant hosts for biosynthesizing methacrylate, which is a useful intermediate for producing methyl methacrylate or poly(methyl methacrylate) esters such as methyl methacrylate or poly(methyl methacrylate). In particular, as described herein, methacrylate can be biosynthetically produced from renewable feedstocks. Production of methacrylate proceeds through isobutyryl CoA. The terms “methacrylate” and “methacrylic acid” are used interchangeably herein to refer to the same compound in any of its neutral or ionized forms, including any salt forms thereof. It is understood by those skilled in the art that the specific form will depend on pH.

In one aspect, this document features a method of producing methacrylate. The method includes enzymatically synthesizing methacryloyl-CoA from pyruvate, and enzymatically converting methacryloyl-CoA to methacrylate using a thioesterase or a CoA-transferase. The thioesterase can be classified under EC 3.1.2.-, and can be the gene product of tesB or YciA. The CoA-transferase can be classified under EC 2.8.3.- and can have Genbank accession number CAB77207.1 (SEQ ID NO:4).

This document also features a method of producing methacrylate. The method includes enzymatically synthesizing isobutyraldehyde from pyruvate, and enzymatically converting isobutryaldehyde to methacrylate. Isobutryaldehyde can be converted to isobutyrate using a phenylacetaldehyde dehydrogenase, isobutyraldehyde dehydrogenase, or lactaldehyde dehydrogenase, isobutyrate can be converted to isobutyryl-CoA using a CoA transferase or a ligase, isobutyryl-CoA can be converted to methacryloyl-CoA using a dehydrogenase, and methacryloyl-CoA can be converted to methacrylate using a thioesterase or CoA transferase. The phenylacetaldehyde dehydrogenase can be classified under EC 1.2.1.39. For example, the phenylacetaldehyde dehydrogenase can have at least 70% sequence identity to the amino acid sequence set forth in SEQ ID NO:8. The lactaldehyde dehydrogenase can be classified under EC 1.2.1.22. The isobutyraldehyde dehydrogenase can have at least 70% sequence identity to the amino acid sequence set forth in SEQ ID NO:6. The CoA transferase can be a propionate CoA transferase classified under EC 2.8.3.1. For example, the propionate CoA-transferase can have at least 70% sequence identity to the amino acid sequence of SEQ ID NO:4 or SEQ ID NO:5. The ligase can be a propionate CoA ligase classified under EC 6.2.1.13.

In one aspect, this document features a method of producing methacrylate. The method includes enzymatically converting methacryloyl-CoA to methacrylate using a thioesterase or CoA-transferase. The thioesterase can be classified under EC 3.1.2.-. For example, the thioesterase can be the gene product of tesB or YciA. The thioesterase can have at least 70% sequence identity to the amino acid sequence of SEQ ID NOs: 1, 2, or 3. The CoA-transferase can be classified under EC 2.8.3.-. For example, the CoA-transferase can have at least 70% sequence identity to the amino acid sequence of SEQ ID NO:4 or SEQ ID NO:5. Methacryloyl-CoA can be enzymatically synthesized from pyruvate. For example, methacryloyl-CoA can be enzymatically synthesized from pyruvate via isobutyraldehyde using, for example, one or more of the following enzymes: an acetolactate synthase; a dihydroxyisovalerate dehydrogenase; a 2,3-dihydroxyisovalerate dehydratase; a 2-oxovalerate decarboxylase; a phenylacetaldehyde dehydrogenase, isobutyraldehyde dehydrogenase or a lactaldehyde dehydrogenase; or a CoA transferase or a ligase; or an acyl-CoA dehydrogenase. Methacryloyl-CoA also can be enzymatically synthesized from pyruvate via conversion of 2-oxo-isovalerate to isobutyryl-CoA, using for example, one or more of the following enzymes: an acetolactate synthase; a dihydroxyisovalerate dehydrogenase; a 2,3-dihydroxyisovalerate dehydratase; a branch chain dehydrogenase; or an acyl-CoA dehydrogenase. The ligase can be a propionate CoA ligase classified under EC 6.2.1.13. The acyl-CoA dehydrogenase can be an isobutyryl-CoA dehydrogenase, e.g., an isobutyryl-CoA dehydrogenase having at least 70% sequence identity to the amino acid sequence set forth in SEQ ID NO:7 or SEQ ID NO:9.

Any of the methods described herein can be performed in a recombinant host (e.g., a prokaryote or a eukaryote). The host can be subjected to a cultivation strategy under anaerobic, aerobic or micro-aerobic cultivation conditions. The host can be cultured under conditions of nutrient limitation either via nitrogen, phosphate or oxygen limitation. The host cells can be retained using ceramic membranes to maintain a high cell density during fermentation. The principal carbon source fed to the fermentation can derive from biological or non-biological feedstocks. The biological feedstock can be, or can derive from, monosaccharides, disaccharides, lignocellulose, hemicellulose, cellulose, lignin such as levulinic acid and furfural, lignin, triglycerides such as glycerol and fatty acids, agricultural waste or municipal waste. The non-biological feedstock can be, or can derive from, natural gas, syngas, CO₂/H₂, methanol, ethanol, non-volatile residue (NVR) or caustic wash waste stream from cyclohexane oxidation processes.

In any of the methods described herein, the host's tolerance to high concentrations of methacrylic acid can be improved through continuous cultivation in a selective environment.

In any of the methods described herein, the endogenous degradation pathways of central metabolites and central precursors leading to and including methacrylic acid can be attenuated in the host.

In any of the methods described herein, the efflux of methacrylic acid across the cell membrane to the extracellular media can be enhanced or amplified by genetically engineering structural modifications to the cell membrane or increasing any associated transporter activity for methacrylic acid.

This document also features a recombinant host comprising at least one exogenous nucleic acid encoding a phenylacetaldehyde dehydrogenase, wherein the recombinant host produces methacrylic acid. The phenylacetaldehyde dehydrogenase can be classified under EC 1.2.1.39. The host further can include an exogenous nucleic acid encoding a propionate CoA transferase. The host further can include an exogenous nucleic acid encoding a propionate CoA ligase. The host further can include an exogenous nucleic acid encoding the gene product of YciA or tesB.

This document also features a recombinant host that includes at least one exogenous nucleic acid encoding a phenylacetaldehyde dehydrogenase, wherein the recombinant host produces methacrylic acid. The phenylacetaldehyde dehydrogenase can be classified under EC 1.2.1.39. For example, the phenylacetaldehyde dehydrogenase can have at least 70% sequence identity to the amino acid sequence set forth in SEQ ID NO:8. The host further can include one or more (e.g., one, two, three, or four) of the following exogenous enzymes: propionate CoA transferase, propionate CoA ligase, thioesterase, and isobutyryl-CoA dehydrogenase. The propionate CoA transferase has at least 70% sequence identity to the amino acid sequence of SEQ ID NO:4 or SEQ ID NO:5. The thioesterase can be the gene product of YciA or tesB. The thioesterase can have has at least 70% sequence identity to the amino acid sequence of SEQ ID NO:1, SEQ ID NO:2, or SEQ ID NO:3. The isobutyryl-CoA dehydrogenase can have at least 70% sequence identity to the amino acid sequence set forth in SEQ ID NO:7 or SEQ ID NO:9.

Any of the recombinant hosts or any of the recombinant hosts used in any of the methods can be a prokaryote. The prokaryote can be from the genus Escherichia such as Escherichia coli; from the genus Clostridia such as Clostridium ljungdahlii, Clostridium autoethanogenum or Clostridium kluyveri; from the genus Corynebacteria such as Corynebacterium glutaniicum; from the genus Cupriavidus such as Cupriavidus necator or Cupriavidus metallidurans; from the genus Pseudomonas such as Pseudomonas fluorescens, Pseudomonas putida or Pseudomonas oleavorans; from the genus Delftia acidovorans, from the genus Bacillus such as Bacillus subtillis; from the genes Lactobacillus such as Lactobacillus delbrueckii; from the genus Lactococcus such as Lactococcus lactis or from the genus Rhodococcus such as Rhodococcus equi.

Any of the recombinant hosts or any of the recombinant hosts used in any of the methods can be a eukaryote. The eukaryote can be from the genus Aspergillus such as Aspergillus niger; from the genus Saccharomyces such as Saccharomyces cerevisiae; from the genus Pichia such as Pichia pastoris; from the genus Yarrowia such as Yarrowia lipolytica, from the genus Issatchenkia such as Issathenkia orientalis, from the genus Debaryomyces such as Debaryomyces hansenii, from the genus Arxula such as Arxula adenoinivorans, or from the genus Kluyveromyces such as Kluyveromyces lactis.

The reactions of the pathways described herein can be performed in one or more cell (e.g., host cell) strains (a) naturally expressing one or more relevant enzymes, (b) genetically engineered to express one or more relevant enzymes, or (c) naturally expressing one or more relevant enzymes and genetically engineered to express one or more relevant enzymes. Alternatively, relevant enzymes can be extracted from any of the above types of host cells and used in a purified or semi-purified form. Extracted enzymes can optionally be immobilized to a solid substrate such as the floors and/or walls of appropriate reaction vessels. Moreover, such extracts include lysates (e.g., cell lysates) or partially purified lysates that can be used as sources of relevant enzymes. In the methods provided by the document, all the steps can be performed in cells (e.g., host cells), all the steps can be performed using extracted enzymes, or some of the steps can be performed in cells and others can be performed using extracted enzymes. In any of the methods, the reaction may be a single step conversion in which one compound is directly converted to a different compound of interest (e.g., methacryloyl-CoA to methacrylate), or the conversion may include two or more steps to convert one compound to a different compound of interest.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims. The word “comprising” in the claims may be replaced by “consisting essentially of” or with “consisting of,” according to standard practice in patent law.

### DESCRIPTION OF DRAWINGS

FIG. 1 is a schematic overview of biosynthetic pathways leading to the production of methacrylic acid.

FIG. 2 contains the amino acid sequences of an Escherichia coli thioesterase (GenBank Accession No. AAA24665.1, SEQ ID NO:1), an Escherichia coli thioesterase (GenBank Accession No. ACX40038, SEQ ID NO:2), a Haemophilus influenzae acyl-CoA thioesterase (GenBank Accession No. AD096882, SEQ ID NO:3), a Clostridium propionicum propionate CoA-transferase (GenBank Accession No. CAB77207.1, SEQ ID NO:4), a Megasphaera elsdenii DSM 20460 propionate CoA-transferase (Genbank Accession No. CCC72964.1, SEQ ID NO:5), a Pseudomonas sp. VLB 120 isobutyraldehyde dehydrogenase (Genbank Accession No. AGZ35351.1, SEQ ID NO:6), a Shewanella oneidensis MR-1 isobutyryl-CoA dehydrogenase (SEQ ID NO:7), an Escherichia coli phenylacetaldehyde dehydrogenase (GenBank Accession No. CAA67780.1, SEQ ID NO: 8), and a Streptomyces avermitilis isobutyryl-CoA dehydrogenase (GenBank Accession No. AAD44196.1, SEQ ID NO:9).

### DETAILED DESCRIPTION

In particular, this document provides enzymes, non-natural pathways, cultivation strategies, feedstocks, host microorganisms and attenuations to the host's biochemical network, which can be used to synthesize methacrylic acid (2-methylpropenoic acid) from central precursors or central metabolites. For example, methacrylic acid can be produced from pyruvate, with synthesis proceeding through 2-oxo-isovalerate followed by isobutyraldehyde, isobutyrate, and isobutyryl CoA. Alternatively, the synthesis can proceed directly from 2-oxo-isovalerate to isobutyryl CoA without first forming isobutyraldehyde and then isobutyrate. As used herein, the term “central precursor” is used to denote any metabolite in a pathway disclosed herein leading to the synthesis of methacrylic acid. The term “central metabolite” is used herein to denote a metabolite that is produced in all microorganisms to support growth.

As such, host microorganisms described herein can include endogenous pathways that can be manipulated such that methacrylic acid can be produced. In an endogenous pathway, the host microorganism naturally expresses all of the enzymes catalyzing the reactions within the pathway. A host microorganism containing an engineered pathway does not naturally express all of the enzymes catalyzing the reactions within the pathway but has been engineered such that all of the enzymes within the pathway are expressed in the host. For example, a host can include endogenous enzymes to synthesize pyruvate. Within an engineered pathway, the enzymes can be from a single source, i.e., from one species, or can be from multiple sources, i.e., different species. Nucleic acids encoding the enzymes described herein have been identified from various organisms and are readily available in publicly available databases such as GenBank or EMBL.

Any of the enzymes described herein that can be used for methacrylic acid production can have at least 70% sequence identity (homology) (e.g., at least 75%, 80%, 85%, 90%, 95%, 97%, 98%, 99%, or 100%) to the amino acid sequence of the corresponding wild-type enzyme. For example, a thioesterase described herein can have at least 70% sequence identity (homology) (e.g., at least 75%, 80%, 85%, 90%, 95%, 97%, 98%, 99%, or 100%) to the amino acid sequence of the Escherichia coli thioesterase such as the gene product of testB (GenBank Accession No. AAA24665.1, SEQ ID NO:1), the Escherichia coli thioesterase of GenBank Accession No. ACX40038 (SEQ ID NO:2), or an acyl-CoA thioesterase from Haemophilus influenzae such as the gene product of yciA (GenBank Accession No. AD096882, SEQ ID NO:3).

For example, a propionate CoA-transferase described herein can have at least 70% sequence identity (homology) (e.g., at least 75%, 80%, 85%, 90%, 95%, 97%, 98%, 99%, or 100%) to the amino acid sequence of the propionate CoA-transferase from Clostridium propionicum (GenBank Accession No. CAB77207.1, SEQ ID NO:4) or Megasphaera elsdenii DSM 20460 (Genbank Accession No. CCC72964.1, SEQ ID NO:5). See, Prabhu et al., 2012, Appl. Environ. Microbiol., 78(24), 8564-8570, which indicates that the propionate CoA-transferase from Megasphaera elsdenii accepts methacryloyl-CoA as a substrate.

For example, an isobutyraldehyde dehydrogenase described herein can have at least 70% sequence identity (homology) (e.g., at least 75%, 80%, 85%, 90%, 95%, 97%, 98%, 99%, or 100%) to the amino acid sequence of the isobutyraldehyde dehydrogenase from Pseudomonas sp. VLB 120 classified under EC 1.2.1.- (Genbank Accession No. AGZ35351.1, SEQ ID NO:6). See, Lang et al., 2014, Microbial Cell Factories, 13(2), 1-14, which indicates that the isobutyraldehyde dehydrogenase accepts isobutyraldehyde as a substrate.

For example, an isobutyryl-CoA dehydrogenase described herein can have at least 70% sequence identity (homology) (e.g., at least 75%, 80%, 85%, 90%, 95%, 97%, 98%, 99%, or 100%) to the amino acid sequence of the isobutyryl-CoA dehydrogenase from Shewanella oneidensis MR-1 classified, for example, under EC 1.3.99.12 (SEQ ID NO:7) or from Streptomyces avermitilis (Genbank Accession No. AAD44196.1, SEQ ID NO:9). See Kazakov et al., 2009, J. Bacteriol., 191(1), 52-64, and Zhang et al., 1999, Microbiology, 145, 2323-2334, which indicate that the isobutyryl-CoA dehydrogenases accept isobutyryl-CoA as a substrate.

For example, a phenylacetaldehyde dehydrogenase described herein can have at least 70% sequence identity (homology) (e.g., at least 75%, 80%, 85%, 90%, 95%, 97%, 98%, 99%, or 100%) to the amino acid sequence of the phenylacetaldehyde dehydrogenase from Escherichia coli (Genbank Accession No. CAA67780.1, SEQ ID NO:8). See, Xiong et al., 2012, Sci. Rep., 2, 1-13, which indicates that the phenylacetaldehyde dehydrogenase accepts isobutyraldehyde as a substrate.

The percent identity (homology) between two amino acid sequences can be determined as follows. First, the amino acid sequences are aligned using the BLAST 2 Sequences (Bl2seq) program from the stand-alone version of BLASTZ containing BLASTP version 2.0.14. This stand-alone version of BLASTZ can be obtained from Fish & Richardson's web site (e.g., www.fr.com/blast/) or the U.S. government's National Center for Biotechnology Information web site (www.ncbi.nlm.nih.gov). Instructions explaining how to use the Bl2seq program can be found in the readme file accompanying BLASTZ. Bl2seq performs a comparison between two amino acid sequences using the BLASTP algorithm. To compare two amino acid sequences, the options of Bl2seq are set as follows: -i is set to a file containing the first amino acid sequence to be compared (e.g., C:\seq1.txt); -j is set to a file containing the second amino acid sequence to be compared (e.g., C:\seq2.txt); -p is set to blastp; -o is set to any desired file name (e.g., C:\output.txt); and all other options are left at their default setting. For example, the following command can be used to generate an output file containing a comparison between two amino acid sequences: C:\Bl2seq c:\seq1.txt -j c:\seq2.txt -p blastp -o c:\output.txt. If the two compared sequences share homology (identity), then the designated output file will present those regions of homology as aligned sequences. If the two compared sequences do not share homology (identity), then the designated output file will not present aligned sequences. Similar procedures can be following for nucleic acid sequences except that blastn is used.

Once aligned, the number of matches is determined by counting the number of positions where an identical amino acid residue is presented in both sequences. The percent identity (homology) is determined by dividing the number of matches by the length of the full-length polypeptide amino acid sequence followed by multiplying the resulting value by 100. It is noted that the percent identity (homology) value is rounded to the nearest tenth. For example, 78.11, 78.12, 78.13, and 78.14 is rounded down to 78.1, while 78.15, 78.16, 78.17, 78.18, and 78.19 is rounded up to 78.2. It also is noted that the length value will always be an integer.

It will be appreciated that a number of nucleic acids can encode a polypeptide having a particular amino acid sequence. The degeneracy of the genetic code is well known to the art; i.e., for many amino acids, there is more than one nucleotide triplet that serves as the codon for the amino acid. For example, codons in the coding sequence for a given enzyme can be modified such that optimal expression in a particular species (e.g., bacteria or fungus) is obtained, using appropriate codon bias tables for that species.

Functional fragments of any of the enzymes described herein can also be used in the methods of the document. The term “functional fragment” as used herein refers to a peptide fragment of a protein that has at least 25% (e.g., at least: 30%; 40%; 50%; 60%; 70%; 75%; 80%; 85%; 90%; 95%; 98%; 99%; 100%; or even greater than 100%) of the activity of the corresponding mature, full-length, wild-type protein. The functional fragment can generally, but not always, be comprised of a continuous region of the protein, wherein the region has functional activity.

This document also provides (i) functional variants of the enzymes used in the methods of the document and (ii) functional variants of the functional fragments described above. Functional variants of the enzymes and functional fragments can contain additions, deletions, or substitutions relative to the corresponding wild-type sequences. Enzymes with substitutions will generally have not more than 50 (e.g., not more than one, two, three, four, five, six, seven, eight, nine, ten, 12, 15, 20, 25, 30, 35, 40, or 50) amino acid substitutions (e.g., conservative substitutions). This applies to any of the enzymes described herein and functional fragments. A conservative substitution is a substitution of one amino acid for another with similar characteristics. Conservative substitutions include substitutions within the following groups: valine, alanine and glycine; leucine, valine, and isoleucine; aspartic acid and glutamic acid; asparagine and glutamine; serine, cysteine, and threonine; lysine and arginine; and phenylalanine and tyrosine. The nonpolar hydrophobic amino acids include alanine, leucine, isoleucine, valine, proline, phenylalanine, tryptophan and methionine. The polar neutral amino acids include glycine, serine, threonine, cysteine, tyrosine, asparagine and glutamine. The positively charged (basic) amino acids include arginine, lysine and histidine. The negatively charged (acidic) amino acids include aspartic acid and glutamic acid. Any substitution of one member of the above-mentioned polar, basic or acidic groups by another member of the same group can be deemed a conservative substitution. By contrast, a nonconservative substitution is a substitution of one amino acid for another with dissimilar characteristics.

Deletion variants can lack one, two, three, four, five, six, seven, eight, nine, ten, 11, 12, 13, 14, 15, 16, 17, 18, 19, or 20 amino acid segments (of two or more amino acids) or non-contiguous single amino acids. Additions (addition variants) include fusion proteins containing: (a) any of the enzymes described herein or a fragment thereof; and (b) internal or terminal (C or N) irrelevant or heterologous amino acid sequences. In the context of such fusion proteins, the term “heterologous amino acid sequences” refers to an amino acid sequence other than (a). A heterologous sequence can be, for example a sequence used for purification of the recombinant protein (e.g., FLAG, polyhistidine (e.g., hexahistidine), hemagglutinin (HA), glutathione-S-transferase (GST), or maltosebinding protein (MBP)). Heterologous sequences also can be proteins useful as detectable markers, for example, luciferase, green fluorescent protein (GFP), or chloramphenicol acetyl transferase (CAT). In some embodiments, the fusion protein contains a signal sequence from another protein. In certain host cells (e.g., yeast host cells), expression and/or secretion of the target protein can be increased through use of a heterologous signal sequence. In some embodiments, the fusion protein can contain a carrier (e.g., KLH) useful, e.g., in eliciting an immune response for antibody generation) or ER or Golgi apparatus retention signals. Heterologous sequences can be of varying length and in some cases can be a longer sequences than the full-length target proteins to which the heterologous sequences are attached.

Engineered hosts can naturally express none or some (e.g., one or more, two or more, three or more, four or more, five or more, or six or more) of the enzymes of the pathways described herein. Endogenous genes of the engineered hosts also can be disrupted to prevent the formation of undesirable metabolites or prevent the loss of intermediates in the pathway through other enzymes acting on such intermediates. Engineered hosts can be referred to as recombinant hosts or recombinant host cells. Thus, as described herein recombinant hosts can include nucleic acids encoding one or more of an acetolactate synthase, a dehydrogenase such as a dihydroxyisovalerate dehydrogenase, a 2-methylacyl-CoA dehydrogenase, a phenylacetaldehyde dehydrogenase, an isobutyraldehyde dehydrogenase, a short-chain or medium chain acyl-CoA dehydrogenase such as an isobutyryl-CoA dehydrogenase, a 2,3-dihydroxyisovalerate dehydratase, a transferase such as a propionate CoA transferase, a ligase such as a acetate CoA ligase, or an acyl-CoA hydrolase or thioesterase as described in more detail below.

In addition, the production of methacrylic acid can be performed in vitro using the isolated enzymes described herein, using a lysate (e.g., a cell lysate) from a host microorganism as a source of the enzymes, or using a plurality of lysates from different host microorganisms as the source of the enzymes.

As described herein, the carbon-carbon double bond of methacrylate can be enzymatically formed by a dehydrogenase such as an enzyme classified, for example, under EC 1.3.8.1, EC 1.3.8.7, EC 1.3.99.12, or other enzymes in the class EC 1.3.99.- as described herein.

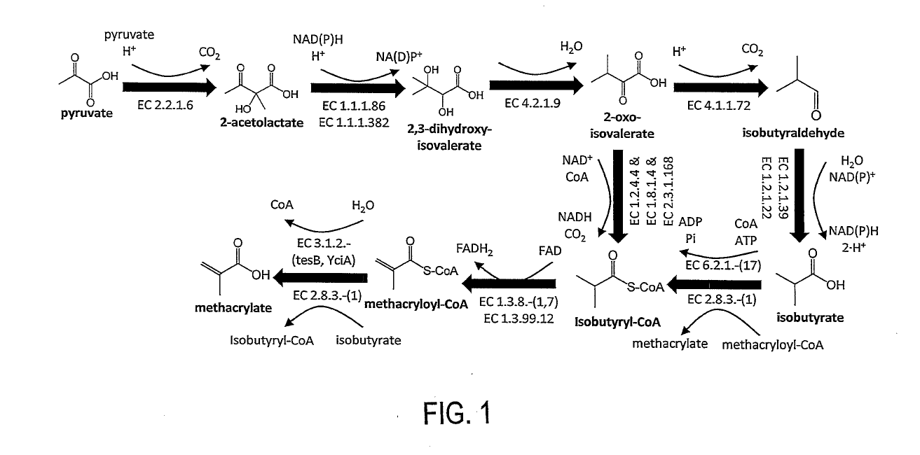

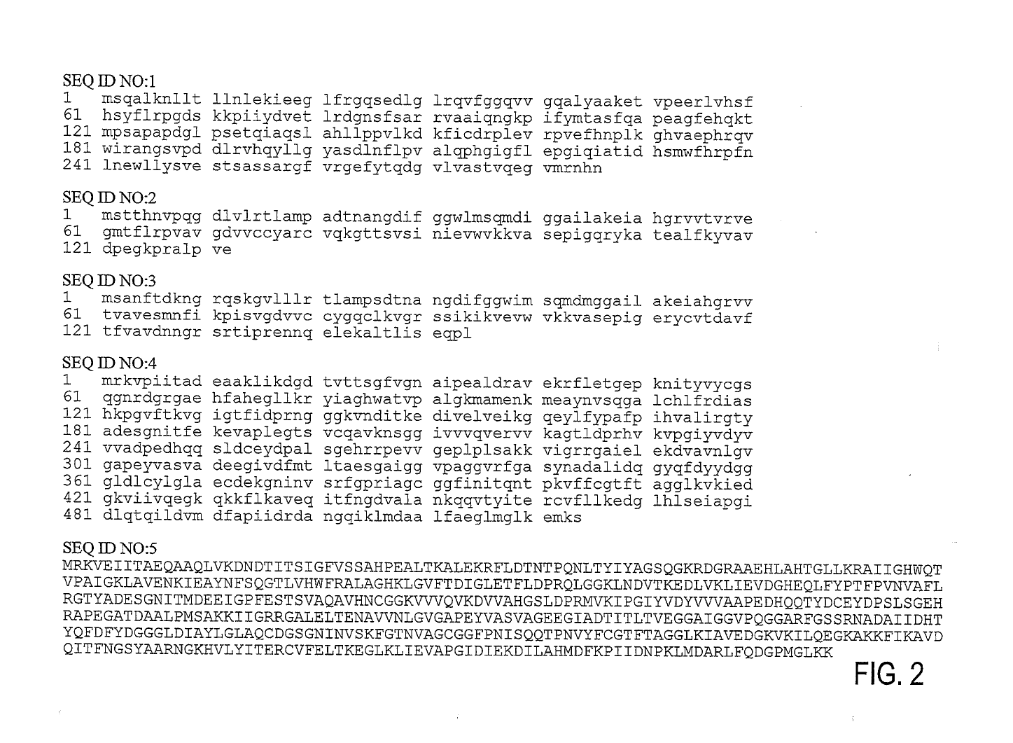

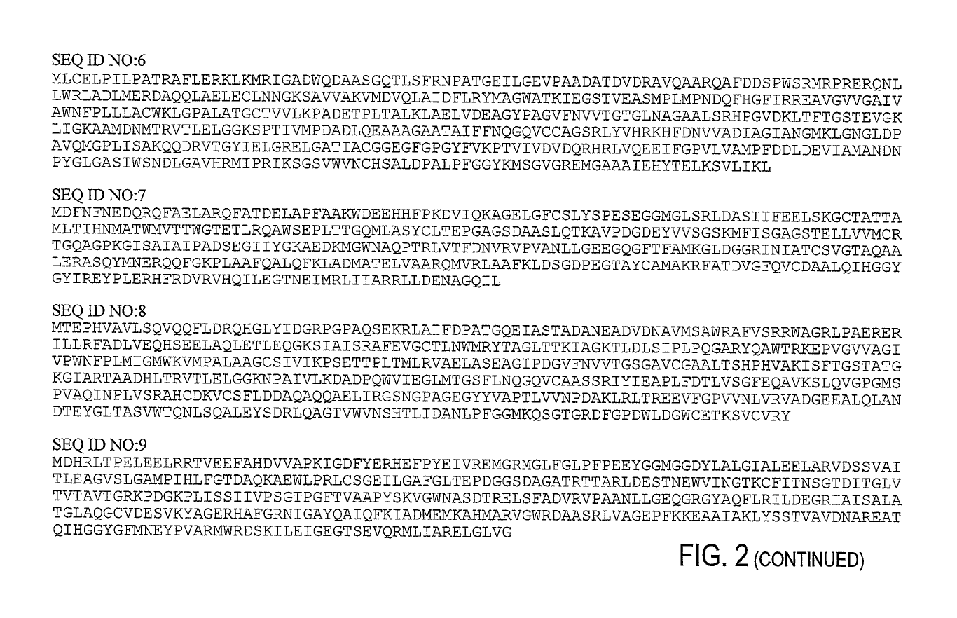

In some embodiments, pyruvate is a precursor to methacrylic acid synthesis. As depicted in FIG. 1, pyruvate can be converted to 2-acetolactate by an acetolactate synthase classified, for example, under EC 2.2.1.6. 2-acetolactate can be converted to 2,3-dihydroxyisovalerate by a dihydroxyisovalerate dehydrogenase classified, for example, under EC 1.1.1.86 or EC 1.1.1.382; followed by conversion of 2,3-dihydroxyisovalerate to 2-oxo-isovalerate by a 2,3-dihydroxyisovalerate dehydratase classified, for example, under EC 4.2.1.9; followed by conversion of 2-oxo-isovalerate to isobutyraldehyde by a 2-oxovalerate decarboxylase classified, for example, under EC 4.1.1.72; followed by conversion of isobutyraldehyde to isobutyrate by a phenylacetaldehyde dehydrogenase classified, for example, under EC 1.2.1.39, an isobutyraldehyde dehydrogenase, or a lactaldehyde dehydrogenase classified, for example, under EC 1.2.1.22; followed by conversion of isobutyrate to isobutyryl-CoA by a CoA transferase classified, for example, under EC 2.8.3.- such as a propionate CoA transferase classified, for example, under EC 2.8.3.1 or by a ligase classified, for example, under EC 6.2.1. such as a propionate CoA ligase classified, for example, under EC 6.2.1.7; followed by conversion of isobutyryl-CoA to methacryloyl CoA by an acyl-CoA dehydrogenase classified, for example, under EC 1.3.8.- such as a short-chain acyl-CoA dehydrogenase classified under EC 1.3.8.1, or a medium-chain acyl-CoA dehydrogenase classified under EC 1.3.8.7, or by a 2-methylacyl-CoA dehydrogenase classified, for example, under EC 1.3.99.12; followed by conversion of methacryloyl CoA to methacrylic acid by, for example, an acyl-CoA hydrolase or thioesterase classified, for example, under EC 3.1.2.- such as the gene product of yciA or tesB or a CoA-transferase classified, for example under EC 2.8.3.-, such as a propionate CoA transferase classified, for example, under EC 2.8.3.1.

In some embodiments, pyruvate is again a precursor to methacrylic acid synthesis but a partially different pathway is used. As depicted in FIG. 1, pyruvate can be converted to 2-acetolactate by an acetolactate synthase classified, for example, under EC 2.2.1.6. 2-acetolactate can be converted to 2,3-dihydroxyisovalerate by a dihydroxyisovalerate dehydrogenase classified, for example, under EC 1.1.1.86 or EC 1.1.1.382; followed by conversion of 2,3-dihydroxyisovalerate to 2-oxo-isovalerate by a 2,3-dihydroxyisovalerate dehydratase classified, for example, under EC 4.2.1.9; followed by conversion of 2-oxo-isovalerate to isobutyryl-CoA by the branch chain dehydrogenase complex classified in its subunits, for example, under EC 1.2.4.4, EC 1.8.1.4 and EC 2.3.1.168; followed by conversion of isobutyryl-CoA to methacryloyl CoA by an acyl-CoA dehydrogenase classified, for example, under EC 1.3.8.- such as a short-chain acyl-CoA dehydrogenase classified under EC 1.3.8.1, or a medium-chain acyl-CoA dehydrogenase classified under EC 1.3.8.7, or by a 2-methylacyl-CoA dehydrogenase classified, for example, under EC 1.3.99.12; followed by conversion of methacryloyl-CoA to methacrylic acid by, for example, an acyl-CoA hydrolase or thioesterase classified, for example, under EC 3.1.2.- such as the gene product of yciA or tesB or CoA-transferase classified, for example under EC 2.8.3.-, such as a propionate CoA transferase classified, for example, under EC 2.8.3.1 (see, for example, the transferase under GenBank Accession No. CAB77207.1, SEQ ID NO:4).

### Cultivation Strategies

In some embodiments, methacrylate can be biosynthesized in a recombinant host using a fermentation strategy that can include anaerobic, micro-aerobic or aerobic cultivation of the recombinant host.

Pathways in the synthesis of methacrylate that incorporate enzymes requiring molecular oxygen and enzymes characterized in vitro as being oxygen sensitive require a micro-aerobic cultivation strategy maintaining a low dissolved oxygen concentration, whilst maintaining sufficient oxygen transfer to prevent substrate oxidation controlled conditions (Chayabatra & Lu-Kwang, Appl. Environ. Microbiol., 2000, 66(2), 493 0 498).

In some embodiments, a cell retention strategy using, for example, ceramic membranes can be employed to achieve and maintain a high cell density during either fed-batch or continuous fermentation in the synthesis of methacrylate.

In some embodiments, the principal carbon source fed to the fermentation in the synthesis of methacrylate can derive from biological or non-biological feedstocks.

In some embodiments, the biological feedstock can be, can include, or can derive from monosaccharides, disaccharides, lignocellulose, hemicellulose, cellulose, lignin such as levulinic acid and furfural, lignin, triglycerides such as glycerol and fatty acids, agricultural waste or municipal waste.

The efficient catabolism of crude glycerol stemming from the production of biodiesel has been demonstrated in several microorganisms such as Escherichia coli, Cupriavidus necator, Pseudomonas oleavorans, Pseudomonas putida and Yarrowia lipolytica (Lee et al., Appl. Biochem. Biotechnol., 2012, 166, 1801-1813; Yang et al., Biotechnology for Biofuels, 2012, 5:13; Meijnen et al., Appl. Microbiol. Biotechnol., 2011, 90, 885-893).

The efficient catabolism of lignocellulosic-derived levulinic acid has been demonstrated in several organisms such as Cupriavidus necator and Pseudomonas putida in the synthesis of 3-hydroxyvalerate via the precursor propanoyl-CoA (Jaremko and Yu, Journal of Biotechnology, 2011, 155, 2011, 293-298; Martin and Prather, Journal of Biotechnology, 2009, 139, 61-67).

The efficient catabolism of lignin-derived aromatic compounds such benzoate analogues has been demonstrated in several microorganisms such as Pseudomonas putida, Cupriavidus necator (Bugg et al., Current Opinion in Biotechnology, 2011, 22, 394-400; Perez-Pantoja et al., FEMS Microbiol. Rev., 2008, 32, 736-794).

The efficient utilization of agricultural waste, such as olive mill waste water has been demonstrated in several microorganisms, including Yarrowia lipolytica (Papanikolaou et al., Bioresour. Technol., 2008, 99(7), 2419-2428).

The efficient utilization of fermentable sugars such as monosaccharides and disaccharides derived from cellulosic, hemicellulosic, cane and beet molasses, cassava, corn and other agricultural sources has been demonstrated for several microorganism such as Escherichia coli, Corynebacterium glutamicum and Lactobacillus delbrueckii and Lactococcus lactis (see, e.g., Hermann et al, Journal of Biotechnology, 2003, 104, 155-172; Wee et al., Food Technol. Biotechnol., 2006, 44(2), 163-172; Ohashi et al., Journal of Bioscience and Bioengineering, 1999, 87(5), 647-654).

The efficient utilization of furfural, derived from a variety of agricultural lignocellulosic sources, has been demonstrated for Cupriavidus necator (Li et al., Biodegradation, 2011, 22, 1215-1225).

In some embodiments, the non-biological feedstock can be or can derive from natural gas, syngas, CO₂/H₂, methanol, ethanol, non-volatile residue (NVR) or a caustic wash waste stream from cyclohexane oxidation processes.

The efficient catabolism of methanol has been demonstrated for the methylotropic yeast Pichia pastoris.

The efficient catabolism of ethanol has been demonstrated for Clostridium kluyveri (Seedorf et al., Proc. Natl. Acad. Sci. USA, 2008, 105(6) 2128-2133).

The efficient catabolism of CO₂ and H₂, which can be derived from natural gas and other chemical and petrochemical sources, has been demonstrated for Cupriavidus necator (Prybylski et al., Energy, Sustainability and Society, 2012, 2:11).

The efficient catabolism of syngas has been demonstrated for numerous microorganisms, such as Clostridium ljungdahlii and Clostridium autoethanogenum (Köpke et al., Applied and Environmental Microbiology, 2011, 77(15), 5467-5475).

The efficient catabolism of the non-volatile residue waste stream from cyclohexane processes has been demonstrated for numerous microorganisms, such as Delftia acidovorans and Cupriavidus necator (Ramsay et al., Applied and Environmental Microbiology, 1986, 52(1), 152-156).

In some embodiments, the host microorganism can be a prokaryote. For example, the prokaryote can be a bacterium from the genus Escherichia such as Escherichia coli; from the genus Clostridia such as Clostridium ljungdahlii, Clostridium autoethanogenum or Clostridium kluyveri; from the genus Corynebacteria such as Corynebacterium glutamicum; from the genus Cupriavidus such as Cupriavidus necator or Cupriavidus metallidurans; from the genus Pseudomonas such as Pseudomonas fluorescens, Pseudomonas putida or Pseudomonas oleavorans; from the genus Delftia such as Delftia acidovorans; from the genus Bacillus such as Bacillus subtillis; from the genus Lactobacillus such as Lactobacillus delbrueckii; or from the genus Lactococcus such as Lactococcus lactis. Such prokaryotes also can be a source of genes to construct recombinant host cells described herein that are capable of producing methacrylic acid.

In some embodiments, the host microorganism can be a eukaryote. For example, the eukaryote can be a filamentous fungus from the genus Aspergillus such as Aspergillus niger. In addition, the eukaryote can be a yeast from the genus Saccharomyces such as Saccharomyces cerevisiae; from the genus Pichia such as Pichia pastoris; or from the genus Yarrowia such as Yarrowia lipolytica; from the genus Issatchenkia such as Issathenkia orientalis; from the genus Debaryomyces such as Debaryomyces hansenii; from the genus Arxula such as Arxula adenoinivorans; or from the genus Kluyveromyces such as Kluyveromyces lactis. Such eukaryotes also can be a source of genes to construct recombinant host cells described herein that are capable of producing methacrylic acid.

Exogenous enzymes that can be expressed in the cells can include one or more of the following: an acetolactate synthase classified, for example, under EC 2.2.1.6; a dehydrogenase classified, for example, under EC 1.1.1.86, EC 1.1.1.382, EC 1.2.1.39, EC 1.3.8.1, EC 1.3.8.7, or EC 1.3.99.12 or the branch chain dehydrogenase complex comprised of EC 1.2.4.4, EC 1.8.1.4 and EC 2.3.1.168; a dehydratase classified, for example, under EC 4.2.1.9; a decarboxylase classified, for example, under EC 4.1.1.72; a CoA transferase classified, for example, under EC 2.8.3.- such as EC 2.8.3.1; a ligase classified, for example, under EC 6.2.1.7; or an acyl-CoA hydrolase or thioesterase classified, for example, under EC 3.1.2.-.

For example, a recombinant host can include at least one exogenous nucleic acid encoding a phenylacetaldehyde dehydrogenase or an isobutyraldehyde dehydrogenase and produce methacrylic acid. The phenylacetaldehyde dehydrogenase can be classified under EC 1.2.1.39, e.g., a phenylacetaldehyde dehydrogenase having at least 70% sequence identity to the amino acid sequence set forth in SEQ ID NO:8. The isobutyraldehyde dehydrogenase can have at least 70% sequence identity to the amino acid sequence set forth in SEQ ID NO:6. The host also can include one or more (e.g., two, three, four, five, six, seven, eight, nine or more) exogenous enzymes that can be used to convert pyruvate to methacrylic acid. For example, a recombinant host can include an exogenous propionate CoA transferase (e.g., a propionate CoA transferase having at least 70% sequence identity to the amino acid sequence of SEQ ID NO:4 or SEQ ID NO:5), an exogenous propionate CoA ligase, an exogenous thioesterase (e.g, the gene product of YciA or tesB or a thioesterase having at least 70% sequence identity to the amino acid sequence of SEQ ID NO:1, SEQ ID NO:2, or SEQ ID NO:3), an exogenous isobutyryl-CoA dehydrogenase (e.g., an isobutyryl-CoA dehydrogenase having at least 70% sequence identity to the amino acid sequence set forth in SEQ ID NO:7 or SEQ ID NO:9), an exogenous acetolactate synthase, an exogenous dihydroxyisovalerate dehydrogenase, a 2,3-dihydroxyisovalerate dehydratase, a 2-oxovalerate decarboxylase, a branch chain dehydrogenase complex.

In some embodiments, substantially pure cultures of recombinant host cells are provided. As used herein, a “substantially pure culture” of a recombinant cell is a culture of that cell in which less than about 40% (i.e., less than about: 35%; 30%; 25%; 20%; 15%; 10%; 5%; 2%; 1%; 0.5%; 0.25%; 0.1%; 0.01%; 0.001%; 0.0001%; or even less) of the total number of viable cells in the culture are viable cells other than the recombinant cells, e.g., bacterial, fungal (including yeast), mycoplasmal, or protozoan cells. The term “about” in this context means that the relevant percentage can be 15% percent of the specified percentage above or below the specified percentage. Thus, for example, about 20% can be 17% to 23%. Such a culture of recombinant cells includes the cells and a growth, storage, or transport medium. Media can be liquid, semi-solid (e.g., gelatinous media), or frozen. The culture includes the cells growing in the liquid or in/on the semi-solid medium or being stored or transported in a storage or transport medium, including a frozen storage or transport medium. The cultures are in a culture vessel or storage vessel or substrate (e.g., a culture dish, flask, or tube or a storage vial or tube).

### Metabolic Engineering

The present document provides methods involving less than all the steps described for the above pathways. Such methods can involve, for example, one, two, three, four, five, six, seven, eight, nine, ten, or more of such steps. Where less than all the steps are included in such a method, the first step can be any one of the steps listed.

Furthermore, recombinant hosts described herein can include any combination of the above enzymes such that one or more of the steps, e.g., one, two, three, four, five, six, seven, eight, nine, ten, or more of such steps, can be performed within a recombinant host.

In addition, this document recognizes that where enzymes have been described as accepting CoA-activated substrates, analogous enzyme activities associated with [acp]-bound substrates exist that are not necessarily in the same enzyme class.

Also, this document recognizes that where enzymes have been described accepting (R)-enantiomers of substrate, analogous enzyme activities associated with (S)-enantiomer substrates exist that are not necessarily in the same enzyme class.

This document also recognizes that where an enzyme is shown to accept a particular co-factor, such as NADPH, or co-substrate, such as acetyl-CoA, many enzymes are promiscuous in terms of accepting a number of different co-factors or co-substrates in catalyzing a particular enzyme activity. Also, this document recognizes that where enzymes have high specificity for e.g., a particular co-factor such as NADH, an enzyme with similar or identical activity that has high specificity for the co-factor NADPH may be in a different enzyme class.

In some embodiments, the enzymes in the pathways outlined above are the result of enzyme engineering via non-direct or rational enzyme design approaches with aims of improving activity, improving specificity, reducing feedback inhibition, reducing repression, improving enzyme solubility, changing stereo-specificity, or changing co-factor specificity.

In some embodiments, the enzymes in the pathways outlined above are gene dosed, i.e., overexpressed, into the resulting genetically modified organism via episomal or chromosomal integration approaches.

In some embodiments, genome-scale system biology techniques such as Flux Balance Analysis are utilized to devise genome scale attenuation or knockout strategies for directing carbon flux to methacrylate.

Attenuation strategies include, but are not limited to; the use of transposons, homologous recombination (double cross-over approach), mutagenesis, enzyme inhibitors and RNAi interference.

In some embodiments, fluxomic, metabolomic and transcriptomal data are utilized to inform or support genome-scale system biology techniques, thereby devising genome scale attenuation or knockout strategies in directing carbon flux to methacrylate.

In some embodiments, the host microorganism's endogenous biochemical network can be attenuated or augmented to (1) ensure the intracellular availability of pyruvate, (2) create an NADH or NADPH imbalance that may only be balanced via the formation of methacrylate, (3) prevent degradation of central metabolites, central precursors leading to methacrylate or (4) to ensure efficient efflux from the cell.

In some embodiments using hosts that naturally accumulate polyhydroxyalkanoates, the polymer synthase enzymes can be attenuated in the host strain.

In some embodiments requiring the intracellular availability of pyruvate, activity of pyruvate carboxylase can be attenuated by a biotin-limiting strategy.

In some embodiments requiring the intracellular availability of 2-acetolactate, feedback inhibition resistant acetolactate synthase enzyme mutants can be overexpressed in the host organism.

In some embodiments requiring an NADH or NADPH imbalance, a FAD reductase is overexpressed in the host strain.

In some embodiments, the endogenous degradation pathways precursors and methacrylate leading to central metabolites and central precursors can be attenuated in the host.

In some embodiments, the efflux of methacrylate across the cell membrane to the extracellular media is enhanced or amplified by genetically engineering structural modifications to the cell membrane or increasing any associated transporter activity for methacrylate.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of producing methacrylate, said method comprising enzymatically synthesizing methacryloyl-CoA from pyruvate via isobutyraldehyde, and enzymatically converting methacryloyl-CoA to methacrylate using a CoA-transferase.

2. The method of claim 1, wherein said CoA-transferase is classified under EC 2.8.3.-.

3. The method of claim 1, wherein said CoA-transferase has at least 70% sequence identity to the amino acid sequence of SEQ ID NO:4 or SEQ ID NO:5.

4. The method of claim 1, wherein methacryloyl-CoA is enzymatically synthesized from pyruvate using one or more of the following enzymes: an acetolactate synthase; a dihydroxyisovalerate dehydrogenase; a 2,3-dihydroxyisovalerate dehydratase; a 2-oxovalerate decarboxylase; a phenylacetaldehyde dehydrogenase, isobutyraldehyde dehydrogenase or a lactaldehyde dehydrogenase; a CoA transferase or an acyl-CoA dehydrogenase.

5. The method of claim 1, wherein isobutryaldehyde is converted to isobutyrate using a phenylacetaldehyde dehydrogenase, isobutyraldehyde dehydrogenase, or lactaldehyde dehydrogenase; isobutyrate is converted to isobutyryl-CoA using a CoA transferase, isobutyryl-CoA is converted to methacryloyl-CoA using an acyl-CoA dehydrogenase, and methacryloyl-CoA is converted to methacrylic acid using a CoA transferase.

6. The method of claim 4, wherein said phenylacetaldehyde dehydrogenase is classified under EC 1.2.1.39.

7. The method of claim 4, wherein said phenylacetaldehyde dehydrogenase has at least 70% sequence identity to the amino acid sequence set forth in SEQ ID NO:8.

8. The method of claim 4, wherein said isobutyraldehyde dehydrogenase has at least 70% sequence identity to the amino acid sequence set forth in SEQ ID NO:6.

9. The method of claim 4, wherein said CoA transferase is a propionate CoA transferase classified under EC 2.8.3.1.

10. The method of claim 9, wherein said propionate CoA-transferase has at least 70% sequence identity to the amino acid sequence of SEQ ID NO:4 or SEQ ID NO:5.

11. The method of claim 4, wherein said acyl-CoA dehydrogenase is an isobutyryl-CoA dehydrogenase.

12. The method of claim 11, wherein said isobutyryl-CoA dehydrogenase has at least 70% sequence identity to the amino acid sequence set forth in SEQ ID NO:7 or SEQ ID NO:9.

13. The method of claim 1, wherein said method is performed in a recombinant host.

14. The method of claim 13, wherein said host is subjected to a cultivation strategy under anaerobic, aerobic or micro-aerobic cultivation conditions.

15. The method of claim 13, wherein said host is cultured under conditions of nutrient limitation either via nitrogen, phosphate or oxygen limitation.

16. The method according to claim 13, wherein said recombinant host cells are retained using ceramic membranes to maintain a high cell density during fermentation.

17. The method of claim 13, wherein a principal carbon source fed to the fermentation derives from biological or non-biological feedstocks.

18. The method of claim 17, wherein the biological feedstock is, or derives from, monosaccharides, disaccharides, lignocellulose, hemicellulose, cellulose, lignin, levulinic acid, formic acid, triglycerides, glycerol, fatty acids, agricultural waste, condensed distillers' solubles or municipal waste.

19. The method of claim 17, wherein the non-biological feedstock is, or derives from, natural gas, syngas, CO₂/H₂, methanol, ethanol, non-volatile residue (NVR) or caustic wash waste stream from cyclohexane oxidation processes, or terephthalic acid/isophthalic acid mixture waste streams.

20. The method of 13, wherein the host is a prokaryote or a eukaryote.

21. The method of claim 20, wherein the host's tolerance to high concentrations of methacrylic acid is improved through continuous cultivation in a selective environment.

22. The method of claim 20, wherein endogenous degradation pathways of central metabolites and central precursors leading to and including methacrylic acid are attenuated in the host.

23. The method of claim 20, wherein the efflux of methacrylic acid across the cell membrane to the extracellular media is enhanced or amplified by genetically engineering structural modifications to the cell membrane or increasing any associated transporter activity for methacrylic acid.

24. A recombinant host comprising at least one exogenous nucleic acid encoding a phenylacetaldehyde dehydrogenase, wherein said recombinant host produces methacrylic acid.

25. The host of claim 24, wherein said phenylacetaldehyde dehydrogenase is classified under EC 1.2.1.39.

26. The host of claim 24, wherein said phenylacetaldehyde dehydrogenase has at least 70% sequence identity to the amino acid sequence set forth in SEQ ID NO:8.

27. The host of claim 24 further comprising an exogenous propionate CoA transferase.

28. The host of claim 27, wherein said propionate CoA transferase has at least 70% sequence identity to the amino acid sequence of SEQ ID NO:4 or SEQ ID NO:5.

29. The host of claim 24 further comprising an exogenous isobutyryl-CoA dehydrogenase.

30. The host of claim 29, wherein said isobutyryl-CoA dehydrogenase has at least 70% sequence identity to the amino acid sequence set forth in SEQ ID NO:7 or SEQ ID NO:9.

31. A bio-derived, bio-based or fermentation-derived product, wherein said product comprises:
i. a composition comprising at least one bio-derived, bio-based or fermentation-derived compound according to claim 1 or any combination thereof,
ii. a bio-derived, bio-based or fermentation-derived polymer comprising the bio-derived, bio-based or fermentation-derived composition or compound of i., or any combination thereof,
iii. a bio-derived, bio-based or fermentation-derived resin comprising the bio-derived, bio-based or fermentation-derived compound or bio-derived, bio-based or fermentation-derived composition of i. or any combination thereof or the bio-derived, bio-based or fermentation-derived polymer of ii. or any combination thereof,
iv. a molded substance obtained by molding the bio-derived, bio-based or fermentation-derived polymer of ii. or the bio-derived, bio-based or fermentation-derived resin of iii., or any combination thereof,
v. a bio-derived, bio-based or fermentation-derived formulation comprising the bio-derived, bio-based or fermentation-derived composition of i., bio-derived, bio-based or fermentation-derived compound of i., bio-derived, bio-based or fermentation-derived polymer of ii., bio-derived, bio-based or fermentation-derived resin of iii., or bio-derived, bio-based or fermentation-derived molded substance of iv, or any combination thereof, or
vi. a bio-derived, bio-based or fermentation-derived semi-solid or a non-semi-solid stream, comprising the bio-derived, bio-based or fermentation-derived composition of i., bio-derived, bio-based or fermentation-derived compound of i., bio-derived, bio-based or fermentation-derived polymer of ii., bio-derived, bio-based or fermentation-derived resin of iii., bio-derived, bio-based or fermentation-derived formulation of v., or bio-derived, bio-based or fermentation-derived molded substance of iv., or any combination thereof.

32. A non-naturally occurring biochemical network comprising at least one substrate of FIG. 1, at least one exogenous nucleic acid encoding a polypeptide having the activity of at least one enzyme of FIG. 1 and at least one product of FIG. 1.

33. A non-naturally occurring composition, comprising at least one substrate of FIG. 1, at least one exogenous nucleic acid encoding a polypeptide having the activity of at least one enzyme of FIG. 1 and at least one bio-derived product of FIG. 1.


SEQUENCE LISTING (9 sequences)

SEQ ID NO: 1 - protein, 286 aa, Escherichia coli
MSQALKNLLTLLNLEKIEEGLFRGQSEDLGLRQVFGGQVVGQALYAAKETVPEERLVHSFHSYFLRPGDSKKPIIYDVETLRDGNSFSARRVAAIQNGKPIFYMTASFQAPEAGFEHQKTMPSAPAPDGLPSETQIAQSLAHLLPPVLKDKFICDRPLEVRPVEFHNPLKGHVAEPHRQVWIRANGSVPDDLRVHQYLLGYASDLNFLPVALQPHGIGFLEPGIQIATIDHSMWFHRPFNLNEWLLYSVESTSASSARGFVRGEFYTQDGVLVASTVQEGVMRNHN

SEQ ID NO: 2 - protein, 132 aa, Escherichia coli
MSTTHNVPQGDLVLRTLAMPADTNANGDIFGGWLMSQMDIGGAILAKEIAHGRVVTVRVEGMTFLRPVAVGDVVCCYARCVQKGTTSVSINIEVWVKKVASEPIGQRYKATEALFKYVAVDPEGKPRALPVE

SEQ ID NO: 3 - protein, 154 aa, Haemophilus influenzae
MSANFTDKNGRQSKGVLLLRTLAMPSDTNANGDIFGGWIMSQMDMGGAILAKEIAHGRVVTVAVESMNFIKPISVGDVVCCYGQCLKVGRSSIKIKVEVWVKKVASEPIGERYCVTDAVFTFVAVDNNGRSRTIPRENNQELEKALTLISEQPL

SEQ ID NO: 4 - protein, 524 aa, Clostridium propionicum
MRKVPIITADEAAKLIKDGDTVTTSGFVGNAIPEALDRAVEKRFLETGEPKNITYVYCGSQGNRDGRGAEHFAHEGLLKRYIAGHWATVPALGKMAMENKMEAYNVSQGALCHLFRDIASHKPGVFTKVGIGTFIDPRNGGGKVNDITKEDIVELVEIKGQEYLFYPAFPIHVALIRGTYADESGNITFEKEVAPLEGTSVCQAVKNSGGIVVVQVERVVKAGTLDPRHVKVPGIYVDYVVVADPEDHQQSLDCEYDPALSGEHRRPEVVGEPLPLSAKKVIGRRGAIELEKDVAVNLGVGAPEYVASVADEEGIVDFMTLTAESGAIGGVPAGGVRFGASYNADALIDQGYQFDYYDGGGLDLCYLGLAECDEKGNINVSRFGPRIAGCGGFINITQNTPKVFFCGTFTAGGLKVKIEDGKVIIVQEGKQKKFLKAVEQITFNGDVALANKQQVTYITERCVFLLKEDGLHLSEIAPGIDLQTQILDVMDFAPIIDRDANGQIKLMDAALFAEGLMGLKEMKS

SEQ ID NO: 5 - protein, 517 aa, Megasphaera elsdenii DSM 20460
MRKVEIITAEQAAQLVKDNDTITSIGFVSSAHPEALTKALEKRFLDTNTPQNLTYIYAGSQGKRDGRAAEHLAHTGLLKRAIIGHWQTVPAIGKLAVENKIEAYNFSQGTLVHWFRALAGHKLGVFTDIGLETFLDPRQLGGKLNDVTKEDLVKLIEVDGHEQLFYPTFPVNVAFLRGTYADESGNITMDEEIGPFESTSVAQAVHNCGGKVVVQVKDVVAHGSLDPRMVKIPGIYVDYVVVAAPEDHQQTYDCEYDPSLSGEHRAPEGATDAALPMSAKKIIGRRGALELTENAVVNLGVGAPEYVASVAGEEGIADTITLTVEGGAIGGVPQGGARFGSSRNADAIIDHTYQFDFYDGGGLDIAYLGLAQCDGSGNINVSKFGTNVAGCGGFPNISQQTPNVYFCGTFTAGGLKIAVEDGKVKILQEGKAKKFIKAVDQITFNGSYAARNGKHVLYITERCVFELTKEGLKLIEVAPGIDIEKDILAHMDFKPIIDNPKLMDARLFQDGPMGLKK

SEQ ID NO: 6 - protein, 497 aa, Pseudomonas sp. VLB120
MLCELPILPATRAFLERKLKMRIGADWQDAASGQTLSFRNPATGEILGEVPAADATDVDRAVQAARQAFDDSPWSRMRPRERQNLLWRLADLMERDAQQLAELECLNNGKSAVVAKVMDVQLAIDFLRYMAGWATKIEGSTVEASMPLMPNDQFHGFIRREAVGVVGAIVAWNFPLLLACWKLGPALATGCTVVLKPADETPLTALKLAELVDEAGYPAGVFNVVTGTGLNAGAALSRHPGVDKLTFTGSTEVGKLIGKAAMDNMTRVTLELGGKSPTIVMPDADLQEAAAGAATAIFFNQGQVCCAGSRLYVHRKHFDNVVADIAGIANGMKLGNGLDPAVQMGPLISAKQQDRVTGYIELGRELGATIACGGEGFGPGYFVKPTVIVDVDQRHRLVQEEIFGPVLVAMPFDDLDEVIAMANDNPYGLGASIWSNDLGAVHRMIPRIKSGSVWVNCHSALDPALPFGGYKMSGVGREMGAAAIEHYTELKSVLIKL

SEQ ID NO: 7 - protein, 385 aa, Shewanella oneidensis MR-1
MDFNFNEDQRQFAELARQFATDELAPFAAKWDEEHHFPKDVIQKAGELGFCSLYSPESEGGMGLSRLDASIIFEELSKGCTATTAMLTIHNMATWMVTTWGTETLRQAWSEPLTTGQMLASYCLTEPGAGSDAASLQTKAVPDGDEYVVSGSKMFISGAGSTELLVVMCRTGQAGPKGISAIAIPADSEGIIYGKAEDKMGWNAQPTRLVTFDNVRVPVANLLGEEGQGFTFAMKGLDGGRINIATCSVGTAQAALERASQYMNERQQFGKPLAAFQALQFKLADMATELVAARQMVRLAAFKLDSGDPEGTAYCAMAKRFATDVGFQVCDAALQIHGGYGYIREYPLERHFRDVRVHQILEGTNEIMRLIIARRLLDENAGQIL

SEQ ID NO: 8 - protein, 499 aa, Escherichia coli
MTEPHVAVLSQVQQFLDRQHGLYIDGRPGPAQSEKRLAIFDPATGQEIASTADANEADVDNAVMSAWRAFVSRRWAGRLPAERERILLRFADLVEQHSEELAQLETLEQGKSIAISRAFEVGCTLNWMRYTAGLTTKIAGKTLDLSIPLPQGARYQAWTRKEPVGVVAGIVPWNFPLMIGMWKVMPALAAGCSIVIKPSETTPLTMLRVAELASEAGIPDGVFNVVTGSGAVCGAALTSHPHVAKISFTGSTATGKGIARTAADHLTRVTLELGGKNPAIVLKDADPQWVIEGLMTGSFLNQGQVCAASSRIYIEAPLFDTLVSGFEQAVKSLQVGPGMSPVAQINPLVSRAHCDKVCSFLDDAQAQQAELIRGSNGPAGEGYYVAPTLVVNPDAKLRLTREEVFGPVVNLVRVADGEEALQLANDTEYGLTASVWTQNLSQALEYSDRLQAGTVWVNSHTLIDANLPFGGMKQSGTGRDFGPDWLDGWCETKSVCVRY

SEQ ID NO: 9 - protein, 386 aa, Streptomyces avermitilis
MDHRLTPELEELRRTVEEFAHDVVAPKIGDFYERHEFPYEIVREMGRMGLFGLPFPEEYGGMGGDYLALGIALEELARVDSSVAITLEAGVSLGAMPIHLFGTDAQKAEWLPRLCSGEILGAFGLTEPDGGSDAGATRTTARLDESTNEWVINGTKCFITNSGTDITGLVTVTAVTGRKPDGKPLISSIIVPSGTPGFTVAAPYSKVGWNASDTRELSFADVRVPAANLLGEQGRGYAQFLRILDEGRIAISALATGLAQGCVDESVKYAGERHAFGRNIGAYQAIQFKIADMEMKAHMARVGWRDAASRLVAGEPFKKEAAIAKLYSSTVAVDNAREATQIHGGYGFMNEYPVARMWRDSKILEIGEGTSEVQRMLIARELGLVG